(19) United States
(12) Patent Application Publication
(10) Pub. No.: US 2013/0072814 A1
(43) Pub. Date: Mar. 21, 2013

(54) BASAL BODY TEMPERATURE ANALYSIS AND THE METHOD THEREOF

(71) Applicants: Bo-Jau KUO, Taipei City (TW); Ching-Hsiu YANG, Taipei City (TW)

(72) Inventors: Bo-Jau KUO, Taipei City (TW); Ching-Hsiu YANG, Taipei City (TW)

(73) Assignee: National Yang Ming University

(21) Appl. No.: 13/476,623

(22) Filed: May 21, 2012

(30) Foreign Application Priority Data
Sep. 20, 2011 (TW) 100133788

(51) Int. Cl. A61B 5/01

(52) U.S. Cl. 600/549

(57) ABSTRACT

The present invention provides a basal body temperature analysis system. The basal body temperature analysis system comprises an analysis unit, a router, a measurement unit, and a portable electronic device. The analysis unit is established in an internet server. The router is connected with the internet server by internet. The measurement unit comprises a basal body temperature measurement device and a wireless communication module. When the basal body temperature measurement device provides basal body temperature information, the wireless communication module transfers the basal body temperature information to the internet server through the router. After analyzing the basal body temperature information, the analysis unit will transfer an analyzed information to a portable electronic device.

### CROSS-REFERENCE TO RELATED APPLICATIONS

This Non-provisional application claims priority under 35 U.S.C. §119(a) on Patent Application No(s). 100133788 filed in Taiwan, Republic of China Sep. 20, 2011, the entire contents of which are hereby incorporated by reference.

### FIELD OF THE INVENTION

This invention relates to a basal body temperature analysis system, and, more particularly, to a basal body temperature analysis system which is combined with a phone to form a loop via a specific basal body temperature measurement device and an analysis unit established in an internet server, and further transmits relative information to users in real time.

### BACKGROUND OF THE INVENTION

In recent years, the problem of infertility is getting serious. Clinically, detecting the basal body temperature is a suggested way to know the menstrual cycle and then to improve the chance of pregnant. FIG. 1 is shown the information of the menstrual cycle. To detect the basal body temperature is not difficult. People only need to detect the basal body temperature before getting up. However, there are some difficult points during the actual detection.

Firstly, it is suggested to detect the mouth temperature as the basal body temperature. Sometimes, people will fall in sleep again before getting up and forget to write down the temperature.

Secondly, it is a little inconvenient for some people to read the thermometer and record it in the dark light of early morning.

Thirdly, people will forget to detect the basal body temperature when they are in a hurry.

Fourthly, even recording the basal body temperature, people might not know exactly the changes between these temperatures and miss the chance to pregnant.

Finally, the basal body temperature is usually recorded on a notebook and is not easy to be provided to doctors who might need to know the real-time situations.

Furthermore, comparing to general thermometer, a thermometer for detecting basal body temperature needs higher accuracy. General thermometer is not detailed enough to detect slight changes between basal body temperatures and might lead misunderstandings and miss the best chance to pregnant. Therefore, it is essential for clinicians to find an effective solution for these problems.

### SUMMARY OF THE INVENTION

The present invention provides a basal body temperature analysis system which is combined with a cloud system. Moreover, the basal body temperature analysis system is combined with a phone to form a loop via a specific basal body temperature measurement device and an analysis unit established in an internet server, and further transmits relative information to users in real time.

The basal body temperature analysis system of the invention comprises an analysis unit, a router, a measurement unit, and a portable electronic device. The analysis unit is established in an internet server. The router is connected with the internet server by internet. The measurement unit comprises a basal body temperature measurement device and a wireless communication module.

The basal body temperature measurement device is used to measure the basal body temperature of a user for providing basal body temperature information. The wireless communication module is connected with the router and the internet server. When the basal body temperature measurement device provides the basal body temperature information, the wireless communication module transfers the basal body temperature information to the internet server through the router. After analyzing the basal body temperature information, the analysis unit transfers an analyzed information to the portable electronic device.

Besides, the measurement unit further comprises an auto-detecting circuit. When the basal body temperature measurement device provides the basal body temperature information, the auto-detecting circuit drives the wireless communication module to transfer the basal body temperature information to the internet server through the router, and after analyzing the basal body temperature information, the analysis unit transfers the analyzed information to the portable electronic device.

The present invention also provides a method of analyzing basal body temperature, and the steps are following:

Step A. a timer is used to remind the user to measure basal body temperature in a default time;

Step B. a basal body temperature information of a user is measured by a basal body temperature measurement device;

Step C. a auto-detecting circuit drives a wireless communication module to transfer the basal body temperature information to a internet server through the router; and

Step D. after analyzing the basal body temperature information, an analysis unit disposed at the internet server transfers an analyzed information to a portable electronic device.

The invention provides a basal body temperature analysis system and the method thereof which can solve the problems of prior art. The advantages of the invention are as follows:

1. The basal body temperature analysis system of the invention includes a timer that can remind users to perform basal body temperature detection everyday.
2. The basal body temperature analysis system of the invention can automatically detect the wireless, drive the wireless communication module, and transfer the basal body temperature information to the internet server through a router. Users don't need to write down the data by themselves or in a dark light situation.
3. The analysis unit can analyze the basal body temperature data and then transfer the results to the portable electronic device. The analyzed information can be shown as an easy-understood explanation that can provide users the information of their own physiological status. Users don't need to be worried about the misunderstanding of the basal body temperature numbers and miss the opportunity of pregnant.
4. Combining the technique of cloud, the physiological status of users can be real-timed provided to their doctors, professionals, friends or families easily.

These and other features, aspects and advantages of the present disclosure will become better understood with regard to the following description, appended claims, and accompanying drawings.

### BRIEF DESCRIPTION OF THE DRAWINGS

FIG. 1 shows the information of the menstrual cycle.

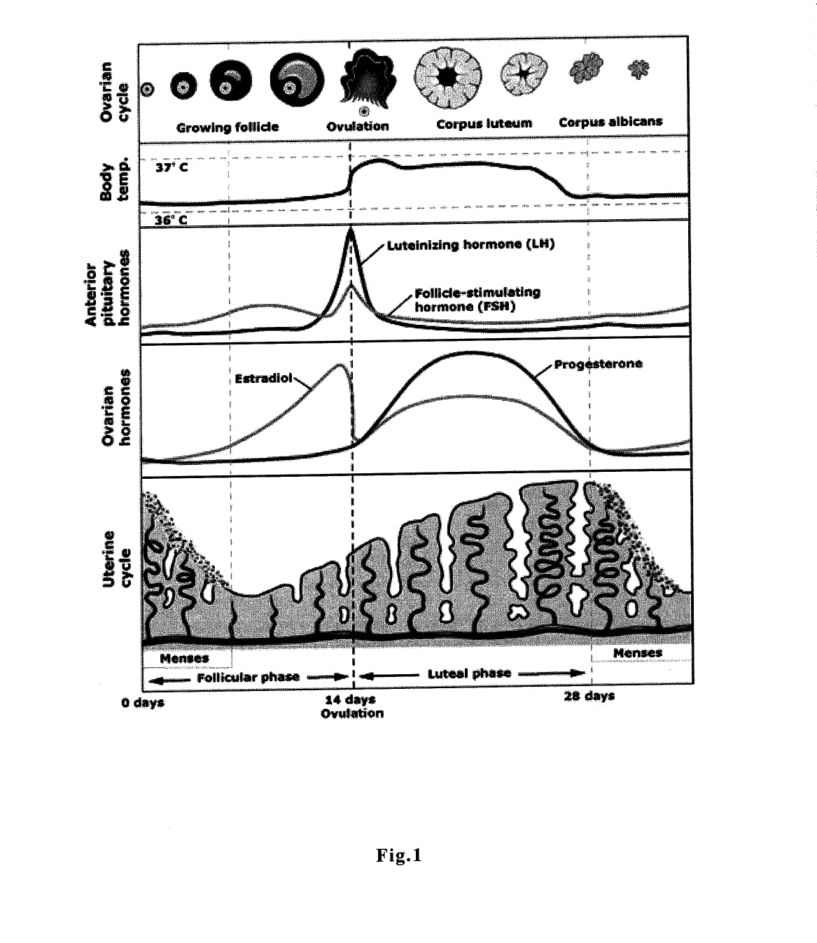

FIG. 2 shows a schematic diagram of the basal body temperature analysis system of the invention.

FIG. 3 shows a schematic diagram of the basal body temperature analysis system of an embodiment.

FIG. 4 shows a flow chart of the method of analyzing basal body temperature in the invention.

### DETAILED DESCRIPTION OF THE INVENTION

For clarity of disclosure, and not by way of limitation, the detailed description of the invention is divided into the subsections that follow.

The present invention provides a basal body temperature analysis system which is combined with a cloud system. Moreover, the basal body temperature analysis system is combined with a phone to form a loop via a specific basal body temperature measurement device and an analysis unit established in an internet server, and further transmits relative information to users in real time.

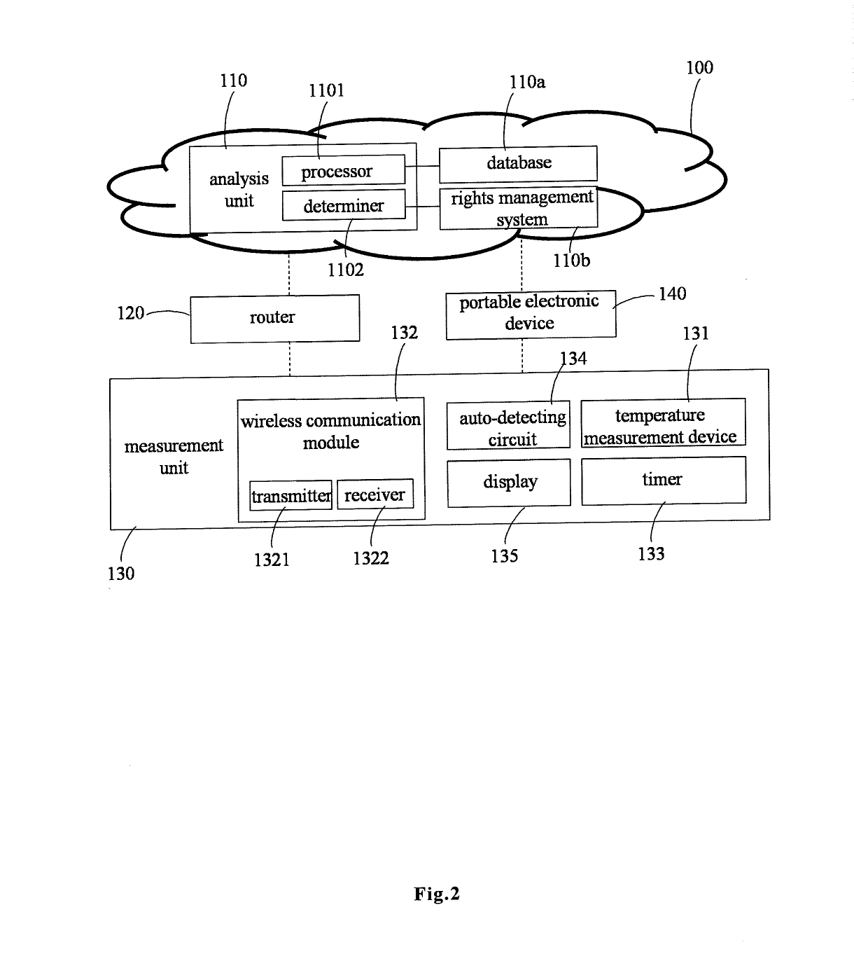

Please refer FIG. 2 showing a schematic diagram of the basal body temperature analysis system of the invention. The basal body temperature analysis system of the invention comprises an analysis unit 110, a router 120, a measurement unit 130, and a portable electronic device 140.

The analysis unit 110 is established in an internet server 100. The router 120 is connected with the internet server 100 by internet. The measurement unit 130 comprises a basal body temperature measurement device 131 and a wireless communication module 132.

Moreover, the basal body temperature measurement device 131 is used to measure the basal body temperature of a user for providing basal body temperature information. The material of the basal body temperature measurement device 131 is composed of metal, silicon, rubber, anti-electrostatic material or the combination of above, but not limited herein.

The measurement unit 130 further comprises a timer 133 to remind the user to measure basal body temperature in a default time. In an embodiment, the timer 133 reminds by transmitting Short Message Service (SMS), Enhanced Message Service (EMS), Multimedia Messaging Service (MMS) or E-mail to the user, but not limited herein.

The wireless communication module 132 is connected with the router 120 and the internet server 100. When the basal body temperature measurement device 131 provides the basal body temperature information, the wireless communication module 132 transfers the basal body temperature information to the internet server 100 through the router 120.

In addition, the wireless communication module 132 comprises a transmitter 1321 and a receiver 1322. The basal body temperature information is received from the basal body temperature measurement device 131 by the receiver 1322, and the basal body temperature information is transferred to the internet server 100 through the router 120 by the transmitter 1321.

The internet server 100 comprises a data base 110a to store all basal body temperature information of the user. The analysis unit 110 comprises a processor 1101 and determiner 1102. The processor 1101 is used to process and analyze the basal body temperature information of the data base. The determiner 1102 is used to compare the basal body temperature information measured at the present day with that before several days, if the compared result is not normal, then the user is reminded. For example, when the basal body temperature information measured at the present day is much higher than that before several days, the determiner 1102 may determine that the present day is the ovulating day, the user will be reminded.

The portable electronic device 140 is connected with the internet server 100. After analyzing the basal body temperature information, the analysis unit 110 transfers an analyzed information to the portable electronic device 140. The portable electronic device 140 can be a phone, a PDA, a personal computer, a laptop computer, or a tablet computer, but not limited herein.

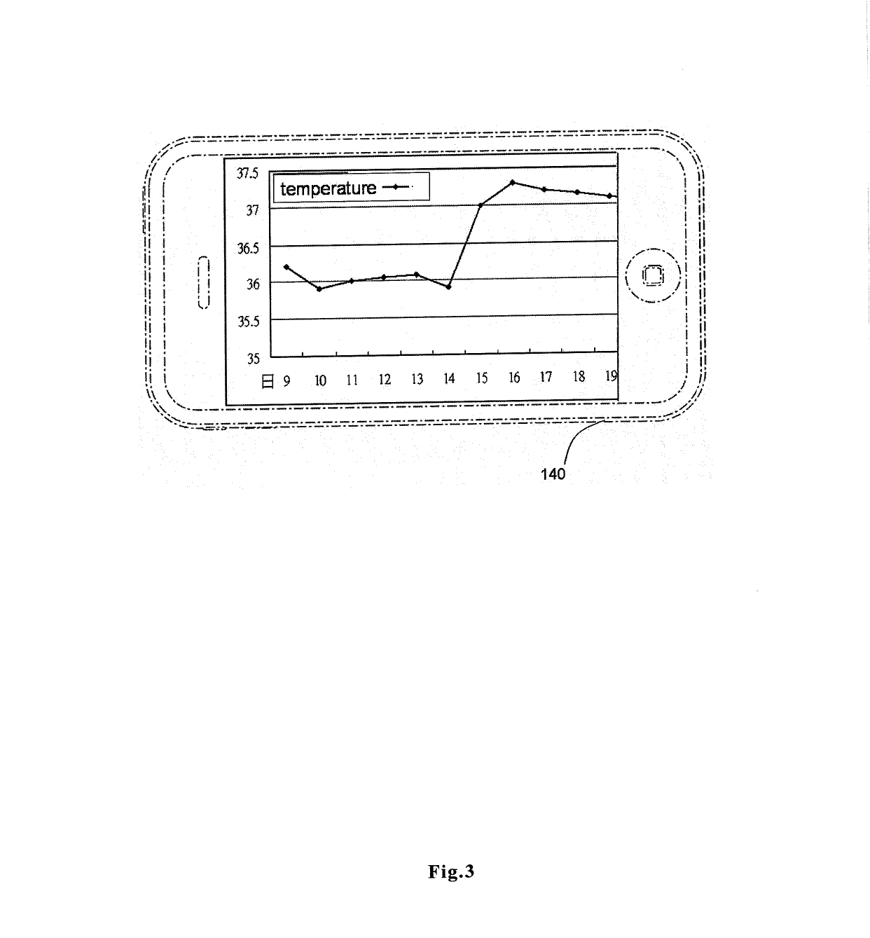

Please refer to FIG. 3 showing a schematic diagram of the basal body temperature analysis system of an embodiment. After analyzing the basal body temperature information, the analysis unit 110 transfers the analyzed information to the portable electronic device 140 via Short Message Service (SMS), Enhanced Message Service (EMS), or Multimedia Messaging Service (MMS), but not limited herein. Moreover, the analyzed information comprises the information of a text, a diagram, a table, a sound, an image or the combination of above, but also not limited herein.

In addition, the analysis unit 110 comprises a rights management system 110b to provide the user authorized to refer the basal body temperature information. As a result, the relative members, such as the user, the user's relatives, friends, doctors or professionals can login the internet server 100 via the rights management system 110b to follow the status and result.

Besides, the measurement unit 130 further comprises an auto-detecting circuit 134 and a display 135.

When the basal body temperature measurement device 131 provides the basal body temperature information, two functions are performed. One function is that the basal body temperature information is shown on the displayl 35. The other function is that the auto-detecting circuit 134 drives the wireless communication module 132 to transfer the basal body temperature information to the internet server 100 through the router 120, and after analyzing the basal body temperature information, the analysis unit 110 transfers the analyzed information to the portable electronic device 140.

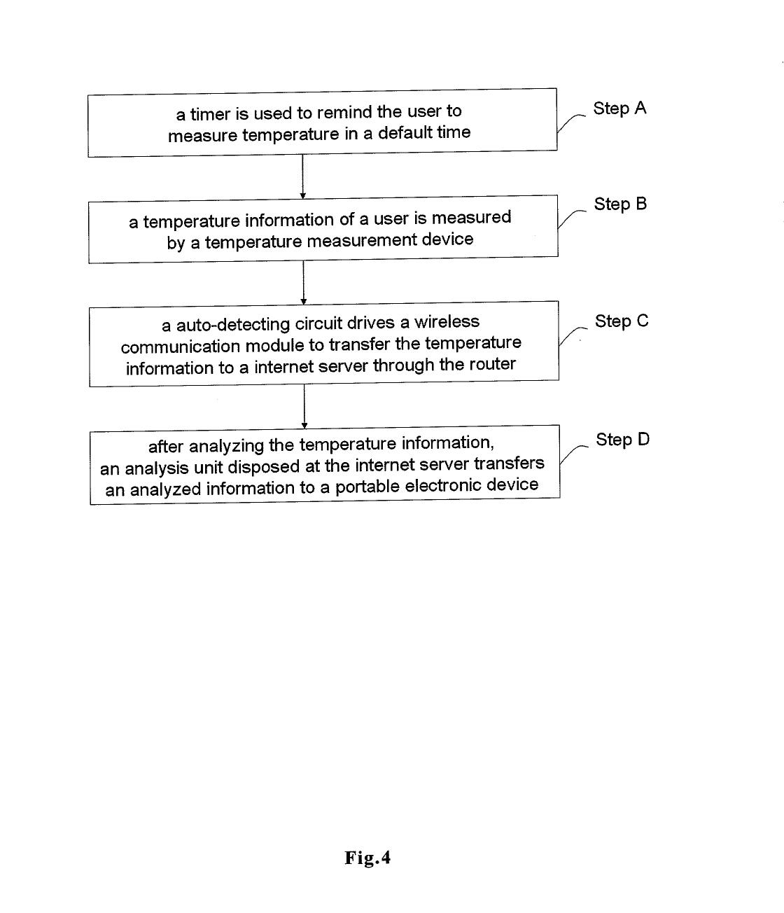

The present invention also provides a method of analyzing basal body temperature. Please refer FIG. 4 showing the flow chart of the method of analyzing basal body temperature in the invention, and the steps are following:

Step A. a timer is used to remind the user to measure basal body temperature in a default time;

Step B. a basal body temperature information of a user is measured by a basal body temperature measurement device;

Step C. a auto-detecting circuit drives a wireless communication module to transfer the basal body temperature information to a internet server through the router; and

Step D. after analyzing the basal body temperature information, an analysis unit disposed at the internet server transfers an analyzed information to a portable electronic device.

In an embodiment, the analyzed information is transferred to the portable electronic device via Short Message Service (SMS), Enhanced Message Service (EMS), or Multimedia Messaging Service (MMS), or the combination of above, but not limited herein. The analyzed information can comprise the information of a text, a diagram, a table, a sound, an image or the combination of above, but not limited herein.

When the basal body temperature measurement device provides the basal body temperature information, the auto-detecting circuit drives the wireless communication module to transfer the basal body temperature information to the internet server through the router, and after analyzing the basal body temperature information, the analysis unit transfers the analyzed information to the portable electronic device.

The invention provides a basal body temperature analysis system and the method thereof which can solve the problems of prior art. The advantages of the invention are as follows:
1. The basal body temperature analysis system of the invention includes a timer that can remind users to perform basal body temperature detection everyday.
2. The basal body temperature analysis system of the invention can automatically detect the wireless, drive the wireless communication module, and transfer the basal body temperature information to the internet server through a router. Users don't need to write down the data by themselves or in a dark light situation.
3. The analysis unit can analyze the basal body temperature data and then transfer the results to the portable electronic device. The analyzed information can be shown as an easy-understood explanation that can provide users the information of their own physiological status. Users don't need to be worried about the misunderstanding of the basal body temperature numbers and miss the opportunity of pregnant.
4. Combining the technique of cloud, the physiological status of users can be real-timed provided to their doctors, professionals, friends or families easily.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A basal body temperature analysis system, comprising:
an analysis unit, established in an internet server;
a router connected with the internet server;
a measurement unit, comprising:
a basal body temperature measurement device, used to measure the basal body temperature of a user for providing a basal body temperature information; and
a wireless communication module, connected with the router and the internet server, when the basal body temperature measurement device provides the basal body temperature information, the wireless communication module transfers the basal body temperature information to the internet server through the router; and
a portable electronic device, connected with the internet server, after analyzing the basal body temperature information, the analysis unit transfers an analyzed information to the portable electronic device.

2. The basal body temperature analysis system according to claim 1, wherein the wireless communication module comprises a transmitter and a receiver.

3. The basal body temperature analysis system according to claim 1, wherein after analyzing the basal body temperature information, the analysis unit transfers the analyzed information to the portable electronic device via Short Message Service (SMS), Enhanced Message Service (EMS), or Multimedia Messaging Service (MMS).

4. The basal body temperature analysis system according to claim 1, wherein the internet server comprises a data base to store the basal body temperature information.

5. The basal body temperature analysis system according to claim 4, wherein the analysis unit comprises a processor to process and analyze the basal body temperature information of the data base.

6. The basal body temperature analysis system according to claim 1, wherein the analyzed information comprises the information of a text, a diagram, a table, a sound, an image or the combination of above.

7. The basal body temperature analysis system according to claim 1, wherein the measurement unit further comprises a timer to remind the user to measure basal body temperature in a default time.

8. The basal body temperature analysis system according to claim 1, wherein the measurement unit further comprises an auto-detecting circuit, when the basal body temperature measurement device provides the basal body temperature information, the auto-detecting circuit drives the wireless communication module to transfer the basal body temperature information to the internet server through the router.

9. The basal body temperature analysis system according to claim 1, wherein the measurement unit further comprises a display, when the basal body temperature measurement device provides the basal body temperature information, the basal body temperature information is shown on the display.

10. The basal body temperature analysis system according to claim 1, wherein the analysis unit comprises a determiner, when the basal body temperature information measured is not normal, the user is reminded.

11. The basal body temperature analysis system according to claim 1, wherein the analysis unit comprises a rights management system to provide the user authorized to refer the basal body temperature information.

12. The basal body temperature analysis system according to claim 1, wherein the material of the basal body temperature measurement device is composed of metal, silicon, rubber, anti-electrostatic material or the combination of above.

13. The basal body temperature analysis system according to claim 1, wherein the portable electronic device is a phone, a PDA, a personal computer, a laptop computer, or a tablet computer.

14. A method of analyzing basal body temperature, comprising:
a. a basal body temperature information of a user is measured by a basal body temperature measurement device;
b. a auto-detecting circuit drives a wireless communication module to transfer the basal body temperature information to a internet server through the router; and
c. after analyzing the basal body temperature information, a analysis unit disposed at the internet server transfers an analyzed information to a portable electronic device.

15. The method of analyzing basal body temperature according to claim 14, wherein before the step a, a timer is used to remind the user to measure basal body temperature in a default time.

16. The method of analyzing basal body temperature according to claim 14, wherein in the step c, the analyzed information is transferred to the portable electronic device via Short Message Service (SMS), Enhanced Message Service (EMS), or Multimedia Messaging Service (MMS).
